(19) United States
(12) Patent Application Publication
(10) Pub. No.: US 2025/0122164 A1
(43) Pub. Date: Apr. 17, 2025

(54) N,N-DIMETHYLAMPHETAMINE ANALOGS FOR TREATING BRAIN DISORDERS

(71) Applicant: The Regents of the University of California, Oakland, CA (US)

(72) Inventors: David E. OLSON, Davis, CA (US); Lee E. DUNLAP, Davis, CA (US)

(21) Appl. No.: 18/707,089

(22) PCT Filed: Nov. 3, 2022
(86) PCT No.: PCT/US2022/079217

§ 371 Date: May 2, 2024

Related U.S. Application Data
(60) Provisional application No. 63/275,146, filed on Nov. 3, 2021.
(60) Provisional application No. 63/299,519, filed on Jan. 14, 2022.
(60) Provisional application No. 63/331,648, filed on Apr. 15, 2022.

(51) Int. Cl. C07D 317/58; A61K 31/36; A61K 45/06; C07C 57/15

(52) U.S. Cl. CPC C07D 317/58; A61K 31/36; A61K 45/06; C07C 57/15

(57) ABSTRACT

The present invention provides R-3,4-methylenedioxy-N,N-dimethylamphetamine, and salts thereof, for treating brain disorders.

### CROSS-REFERENCES TO RELATED APPLICATIONS

This application claims priority to U.S. Provisional Application Nos. 63/275,146, filed Nov. 3, 2021, 63/299,519, filed Jan. 14, 2022, and 63/331,648, filed Apr. 15, 2022, each of which is incorporated herein in its entirety for all purposes.

### BACKGROUND OF THE INVENTION

S,R(+/−)-3,4-methylenedioxymethamphetamine (SR-MDMA) is a substituted phenethylamine with structural similarities to both amphetamines as well as psychedelics. It was first described and synthesized in a German patent issued to Merk in 1912 and largely forgotten till the late 1970s during which the drug was re-discovered by Alexander Shulgin and demonstrated to possess therapeutic potential in talk therapy. Despite its prohibition and purported toxicity, interest in MDMA has persisted; double-blind placebo-controlled studies have demonstrated that SR-MDMA paired psychotherapy sessions lead to dramatic clinical responses in decreasing the symptoms of post-traumatic stress disorder (PTSD), with a response rate in 83% of patients in the SR-MDMA group compared to 25% of those in the placebo group. SR-MDMA is currently in phase 3 clinical trials for use in talk therapy for those with severe PTSD and has demonstrated robust efficacy in lowering clinical measures of PTSD symptoms as well as being well tolerated, even in those with comorbidities.

While the therapeutic potential of SR-MDMA is clear, it's pharmacology also lends itself to be apt for misuse. Similar to amphetamines, SR-MDMA has high affinity for the serotonin transporter (SERT) and dopamine transporter (DAT), leading to release of these monoamines. As a result of its monoamine releasing properties, primarily through the release of dopamine, SR-MDMA has acute reinforcing effects in rodents, which is indicative of abuse potential. The enantio-specific pharmacology has been primarily studied by Howell and coworkers and demonstrated that the S enantiomer of MDMA is primarily responsible for the monoamine releasing properties and subsequently the abuse liability, while the R enantiomer lacks potent monoamine releasing properties and is still able to promote prosocial as well as fear extinction in preclinical rodent models. Both the prosocial as well as fear extinction properties of SR-MDMA have largely been attributed to activity at SERT, and potential downstream serotonin receptor activity as in the case of the prosocial effects being mediated by SERT mediated serotonin release and subsequent activation of 5-HT1B receptors in the nucleus acumbens.

Despite the apparent importance of serotonin release it fails to represent the full repertoire of SR-MDMA's relevant pharmacological activity—it should also be noted that 5-HT release alone is insufficient to promote the enhancement in fear extinction learning—there are a plethora of studies examining SR-MDMA's activity at a variety of neuroreceptors including the 5-HT2B, 5-HT2A, trace amine-associated receptor 1 (TAAR1), and sigma-1 receptors though the majority of which only bind SR-MDMA in the low micromolar range, suggesting that they may lack in vivo relevance depending on the dose. Our lab has previously demonstrated that SR-MDMA is a potent psychoplastogen, capable of promoting neural plasticity in rat cortical cultures in a 5-HT2 dependent fashion, suggesting that some of SR-MDMA's therapeutic properties may stem from activation of 5-HT2 receptors. Furthermore, there is preponderance of evidence that SR-MDMA leads to upregulation of brain derived neurotropic factor (BDNF) in the frontal cortex of mice, suggesting a potential mechanism through frontal cortex mediated plasticity could lead to therapeutic effects in accelerated fear-extinction learning.

In an effort to both develop safer MDMA analogous with decreased abuse potential as well as further the mechanistic understanding of MDMA's behavioral properties the compound R-3,4-Methylenedioxy-N, N-dimethylamphetamine (R-MDDMA) was developed; the racemic mixture originally synthesized and described by Shulgin in PIHKAL: A Chemical Love Story, the R enantiomer being isolated for testing and dubbed LED or R-MDDMA. Based on the improved safety profile of R-MDMA, it is hypothesized that making the small molecular change via an additional methyl group on the primary amine could confer the analog with potentially distinct pharmacology that retains some of the therapeutic properties of R-MDMA while further decreasing the abuse potential. To test this, it was demonstrated that R-MDMA and LED robustly promote plasticity in rat cortical cultures. We then proceeded to assess the safety profile of the R-MDMA and LED using a combination of a locomotor, body temperature, and a head-twitch response assays. Fear-extinction represents one of the major preclinical models in which MDMA displays efficacy and as such R-MDMA and LED were directly compared, showing that both promote extinction learning.

### BRIEF SUMMARY OF THE INVENTION

In one embodiment, the present invention provides a compound of Formula I:

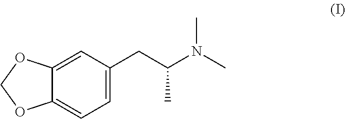

or a pharmaceutically acceptable salt thereof.

In another embodiment, the present invention provides an isolated compound of Formula I:

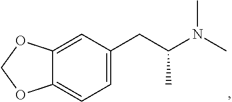

or a pharmaceutically acceptable salt thereof.

In another embodiment, the present invention provides a composition comprising
a compound of Formula I:

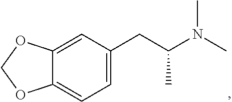

or a pharmaceutically acceptable salt thereof; and
a compound of Formula II:

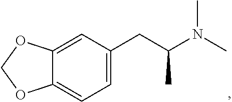

or a pharmaceutically acceptable salt thereof, wherein the compound of Formula II is present in an amount of less than 10% (w/w).

In another embodiment, the present invention provides a method for increasing neural plasticity, comprising contacting a neural cell with a compound of Formula I:

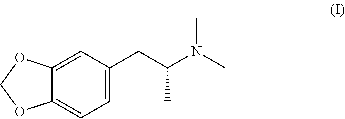

or a pharmaceutically acceptable salt thereof, in an amount sufficient to increase neural plasticity of the neural cell.

In another embodiment, the present invention provides a method of treating a brain disorder, comprising administering to a subject in need thereof a therapeutically effective amount of a compound of Formula I:

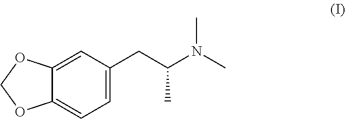

or a pharmaceutically acceptable salt thereof, thereby treating the brain disorder.

In another embodiment, the present invention provides a method for increasing at least one of translation, transcription or secretion of neurotrophic factors, comprising contacting a neural cell with a compound of Formula I:

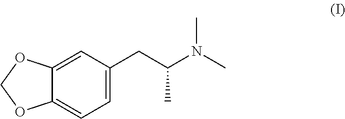

or a pharmaceutically acceptable salt thereof, in an amount sufficient to increase neural plasticity of the neural cell.

In another embodiment, the present invention provides a method for modulating a 5-HT2 receptor, comprising contacting a neural cell with a compound of the present invention, or a pharmaceutically acceptable salt thereof, or a composition of the present invention, in an amount sufficient to modulate the 5-HT2 receptor.

In another embodiment, the present invention provides a method for treating a disease or condition responsive to the modulation of a 5-HT2 receptor, comprising contacting a neural cell with a compound of the present invention, or a pharmaceutically acceptable salt thereof, or a composition of the present invention, in an amount sufficient to treat the disease or condition responsive to modulation of the 5-HT2 receptor.

### BRIEF DESCRIPTION OF THE DRAWINGS

FIG. 1A-C shows R-MDMA and LED promote plasticity. FIG. TA and FIG. 1B show Sholl analysis DIV 6 rat cortical cultures following treatments for 72-hours and then fixed and stained. Nmax values represent the largest amount of branching that occurred and is representative of the complexity of the dendritic arbor of the treatment group. Both LED and R-MDMA cause increases in dendritic complexity at the 10 uM and 100 nM concentrations. N=2 animal with 51-103 cells per treatment group. FIG. 1C shows dendritic spine analysis of DIV 15 rat cortical neurons following treatments for 24-hours and then fixed and stained. Spines/10 uM represents the amount of dendritic spines on a dendrite per 10 um for the treatment group. N=2 animal with 30-37 cells per treatment group. Error bars represent s.e.m *P<0.05, ***P<0.001, ****P<0.0001, as compared to VEH (one-way ANOVA with Dunnett's post-hoc test). VEH=vehicle (0.01% DMSO), R-MDMA=R-3,4-Methylenedioxy methamphetamine, and LED=R-3,4-Methylenedioxy-N,N-dimethylamphetamine (R-MDDMA).

FIG. 2A-E shows R-MDMA and LED safety profiles. FIG. 2A and FIG. 2B show mice were treated with R-MDMA or LED and temperature was measured every 30 minutes for 2-hours. The 50 mg/kg doses of LED and R-MDMA both induced a significant decrease in temperature relative to the vehicle at the 30-minute time point. N=6 mice (3 male and 3 female) per treatment group (two-way ANOVA with Dunnett's multiple comparisons test). FIG. 2C and FIG. 2D show after treatment with R-MDMA or LED the same mice were placed in a novel arena and total distance and time spent in the center were measured for 30-minutes. R-MDMA causes a dose dependent increase in locomotion and all doses decreased time spent in the center while LED causes a dose dependent decrease in locomotion while only the highest doss caused a decrease in time spent in the center (one-way ANOVA with Dunnett's post-hoc test). FIG. 2E shows during the first 20-minutes in the novel area the head twitch response (HTR) was analyzed to assess the hallucinogenic potential of both compounds. Neither compound elicited a significant HTR (one-way ANOVA with Dunnett's post-hoc test). Error bars represent s.e.m *P<0.05, **P<0.01, ***P<0.001, ****P<0.0001. VEH=vehicle (0.9% saline).

FIG. 3A-D shows R-MDMA and LED promote fear extinction. FIG. 3A shows fear extinction procedure. FIG. 3B shows R-MDMA and LED treatment before extinction training reduced conditioned freezing 24-hours later. All treatments were given intraperitoneally 30-min before extinction training on day 2. Both doses of R-MDMA resulted in decreased freezing during extinction training and testing while the 12.5 mg/kg dose of LED resulted in reduced freezing on testing day across 16 CS tones. N=16 mice (8 male and 8 female) per group (two-way ANOVA with Dunnett's multiple comparison test). FIG. 3C shows R-MDMA fear extinction test curve, both R-MDMA curves are statistically lower than vehicle control. FIG. 3D shows LED feat extinction test curve, both LED curves are statistically lower than the vehicle control curve. Error bars represent s.e.m *P<0.05, **P<0.01, ****P<0.0001.

FIG. 4A-D shows R-MDMA and LED mechanism of action. FIG. 4A shows Sholl analysis DIV 6 rat cortical neurons following treatments for 72-hours. KTSN was pretreated 30-minutes prior to the addition of either LED or R-MDMA. The dendritogenesis effects of both LED and R-MDMA are blocked by ketanserin. N=2 animal with 56-74 cells per treatment group FIG. 4B shows dendritic spine analysis of DIV 15 rat cortical neurons following 30-minute KTSN pretreatment followed by treatments for 24-hours. KTSN blocks the spinogenesis effects of both LED and R-MDMA. N=2 animal with 30-37 cells per treatment group. Note this is the same data from FIG. 1C with the addition of KTSN, LED+KTSN, and R-MDMA+KTSN. FIG. 4C shows DIV 10-14 rat cortical neurons expressing the psychlight 5-HT2A biosensor. Neither LED nor R-MDMA induced a significant response in the sensor. N=2 animals with 23-57 cells per treatment group. FIG. 4D shows [3H]5-HT efflux from SERT expressing HEK293T cells treated with increasing concentrations of LED or R-MDMA (10 pM-100 uM). LED lacks serotonin releasing properties while R-MDMA is capable of promoting [3H]5-HT release. Error bars represent s.e.m *P<0.05, ***P<0,001, ****P<0.0001, as compared to VEH (one-way ANOVA with Dunnett's post-hoc test). VEH=vehicle (0.02% DMSO for blocking experiments) and KTSN=ketanserin.

FIG. 5 shows Sholl analysis of cultured cortical neurons indicates that R-MDDMA is more effect at promoting plasticity than either the S-enantiomer or the racemic mixture. Furthermore, it is more effective than racemic MDMA.

FIG. 6 shows that LED (R-MDDMA) produces antidepressant behavioral effects. Mice were administered LED, R-MDMA, the positive control ketamine (KET), or the vehicle control (VEH) via intraperitoneal injection. After 24 h, the animals were subjected to a tail suspension test. *p<0.05 compared to the vehicle control (one-way ANOVA with Dunnett's post hoc test).

### DETAILED DESCRIPTION OF THE INVENTION

### I. General

The present invention provides (R)-1-(benzo[d][1,3]dioxol-5-yl)-N,N-dimethylpropan-2-amine (R-MDDMA or LED), and salts thereof, for treating brain disorders and promoting neural plasticity without the hallucinogenic properties of MDMA.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

Abbreviations. VEH=vehicle; KET=ketamine; IBO=ibogaine; NOR=noribogaine; IBG=ibogainalog; TBG=tabemanthalog; KETSN=ketanserin; SI=sertindole; DOI=2,5-dimethoxy-4-iodoamphetamine; FST=forced swim test; EtOH=ethanol, DMSO=dimethyl sulfoxide, ATP=adenosine triphosphate.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to a compound in salt form, wherein the compound are suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Isomers” refers to compounds with same chemical formula but different connectivity between the atoms in the molecule, leading to distinct chemical structures.

Isomers include structural isomers and stereoisomers. Examples of structural isomers include, but are not limited to tautomers and regioisomers. Examples of stereoisomers include but are not limited to diastereomers and enantiomers.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neural plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses. These changes can appear in neurons, glia, and in other parts of the brain. “Neuronal plasticity” refers to plasticity of the neurons.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Anxiety disorders” are defined in the Diagnostic and Statistical Manual of Mental Disorders, Fifth Edition (DSM-5 2013) as a group of disorders that share features of persistent, excessive fear and anxiety. Anxiety disorders typically have a duration of at least 6 months. In children, anxiety disorders are typically diagnosed using one or more rating scales, such as, the Pediatric Anxiety Rating Scale (PARS), the Children's Depressive Rating Scale, and the Children's Yale-Brown Obsessive Compulsive Scale. Anxiety disorders include, but are not limited to, generalized anxiety disorder, social anxiety disorder, social phobia, panic attack, panic disorder, post-traumatic stress disorder, agoraphobia, separation anxiety disorder, separation anxiety disorder, anxiety disorder induced by a substance/medication or due to another medical condition, and selective mutism, among others.

“Generalized anxiety disorder” is excessive worry about a variety of everyday problems occurring more days than not for at least 6 months. “Social anxiety disorder” is a marked fear or anxiety about one or more social situations and is interchangeable with “social phobia”. “Panic attacks” are the abrupt onset of intense fear or discomfort associated with symptoms such as heart palpitations, sweating, dizziness, or nausea. “Panic disorder” is diagnosed in patients with recurrent unexpected panic attacks. “Post-traumatic stress disorder” (or “PTSD”) is a condition that can occur in people who have been exposed to an actual or threatened death, serious injury, or sexual violence, wherein the individual experiences recurrent distressing memories, flashbacks, psychological distress, and/or physiological reactions to cues following the event. “Agoraphobia” is a condition wherein an individual has marked fear about situations such as being in public spaces, standing in crowds, or being outside of their home alone. “Separation anxiety disorder” is developmentally inappropriate and excessive fear or anxiety concerning separation from those to whom the individual is attached. “Selective mutism” is a condition characterized by consistent failure to speak in specific social situations in which there is an expectation to speak (e.g., in school), despite having the ability to speak in other situations.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” or “NF” refers to a family of soluble peptides or proteins which support the survival, growth, and differentiation of developing and mature neurons. Most NFs exert their trophic effects on neurons by signaling through tyrosine kinases, usually a receptor tyrosine kinase. In the mature nervous system, they promote neuronal survival, induce synaptic plasticity, and modulate the formation of long-term memories. NFs also promote the initial growth and development of neurons in the central nervous system and peripheral nervous system. Some NFs are also released by the target tissue in order to guide the growth of developing axons. According to the invention, the term “neurotrophic factor” encompasses all neurotrophins, growth factors, and other substances that promote survival and repair of the cells of the nervous system.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT2A) are modulators of the receptor.

“Agonism, “agonist” or “agonisin” refers to the activation of a receptor or enzyme by a modulator, or agonist, to produce a biological response. By way of example only, “5HT_{2A} agonist” can be used to refer to a compound that exhibits an EC₅₀ with respect to 5HT_{2A} activity of no more than about 100 μM. In some embodiments, “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism”, “antagonist” or “antagonizing” refers to the inactivation of a receptor or enzyme by a modulator, or antagonist. Antagonism of a receptor, for example, is when a molecule binds to the receptor and does not allow activity to occur. An antagonist has no activity in the absence of an agonist or inverse agonist but can block the activity of either, causing no change in the biological response.

“IC₅₀” refers to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an antagonist or inhibitor) that is required for 50% inhibition of a receptor, for example, 5HT2A.

### III. Compounds

The present invention provides (R)-1-(benzo[d][1,3]dioxol-5-yl)-N,N-dimethylpropan-2-amine, or R-3,4-methylenedioxy-N,N-dimethylamphetamine (R-MDDMA or LED), or pharmaceutically acceptable salts thereof:

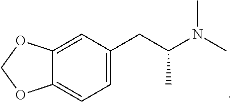

In some embodiments, the present invention provides a compound of Formula I:

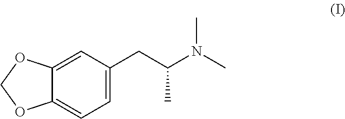

or a pharmaceutically acceptable salt thereof. In some embodiments, the compound of Formula I has the structure:

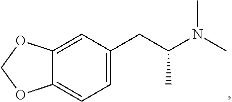

substantially free of a compound of Formula II:

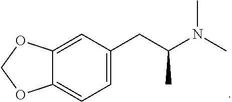

The compounds of the present invention can also be in the salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

In some embodiments, the compound of Formula I comprises hydrochloric acid, hydrobromic acid, hydriodic acid, nitric acid, carbonic acid, monohydrogencarbonic acid, phosphoric acid, monohydrogenphosphoric acid, dihydrogenphosphoric acid, sulfuric acid, monohydrogensulfuric acid, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, glucuronic acid, galactunoric acid, or glutamic acid.

In some embodiments, the compound has the structure:

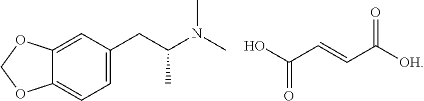

In some embodiments, the present invention provides an isolated compound of Formula I:

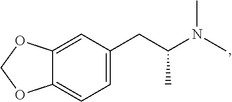

or a pharmaceutically acceptable salt thereof. In some embodiments, the isolated compound is substantially free of a compound of Formula II

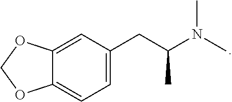

The compound of Formula I can be present in a composition with the compound of Formula II:

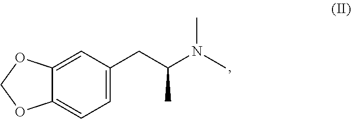

where the compound of Formula II is present in an amount less than 50% (w/w), or less than 45, 40, 35, 30, 25, 20, 15, 10, 9, 8, 7, 6, 5, 4, 3, 2, or less than 1% (w/w). In some embodiments, the present invention provides a compound comprising: a compound of Formula I:

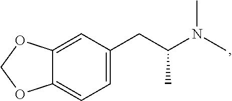

or a pharmaceutically acceptable salt thereof; and a compound of Formula II:

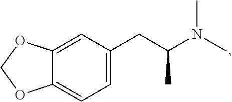

or a pharmaceutically acceptable salt thereof, wherein the compound of Formula II is present in an amount of less than 10% (w/w). In some embodiments, the compound of Formula II is present in an amount of less than 1% (w/w).

The present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of the present invention are useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of this invention can generally be prepared according to the methods known by one of skill in the art by substituting an isotopically-labeled reagent for a non-isotopically labeled reagent. Compounds of the present invention can be isotopically labeled at positions adjacent to the basic amine, in aromatic rings, and the methyl groups of methoxy substituents.

The present invention includes all tautomers and stereoisomers of compounds of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

### IV. Pharmaceutical Compositions and Formulations

In some embodiments, the present invention provides a pharmaceutical composition comprising a compound of the present invention, or a pharmaceutically acceptable salt thereof, and a pharmaceutically acceptable excipient.

The pharmaceutical compositions of the present invention can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J Microencapsul. 13:293-306, 1996; Chonn, Curr. Opin. Biotechnol. 6:698-708, 1995; Ostro, Am. J Hosp. Pharm. 46:1576-1587, 1989).

### V. Administration

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### VI. Methods of Treatment

The compounds of the present invention can be used for increasing neural and neuronal plasticity. The compounds of the present invention can also be used to treat any brain disease. The compounds of the present invention can also be used for increasing at least one of translation, transcription or secretion of neurotrophic factors.

In some embodiments, a compound of the present invention is used to treat a brain disorder. In some embodiments, the compounds have, for example, anti-addictive properties, antidepressant properties, anxiolytic properties, or a combination thereof. In some embodiments, the brain disorder is a neuropsychiatric disease. In some embodiments, the brain disorder is a mood or anxiety disorder. In some embodiments, the brain disorder is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the brain disorder is a migraine or cluster headache. In some embodiments, the brain disorder is a neurodegenerative disorder, Alzheimer's disease, or Parkinson's disease. In some embodiments, the brain disorder is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the brain disorder is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the brain disorder is post-traumatic stress disorder (PTSD), addiction (e.g., substance use disorder), schizophrenia, depression, or anxiety. In some embodiments, the brain disorder is addiction (e.g., substance use disorder). In some embodiments, the brain disorder is depression. In some embodiments, the brain disorder is anxiety. In some embodiments, the brain disorder is post-traumatic stress disorder (PTSD). In some embodiments, the brain disorder is stroke or traumatic brain injury. In some embodiments, the brain disorder is schizophrenia.

In some embodiments, a compound of the present invention is used for increasing neural plasticity. In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the compounds of the present invention have activity as 5-HT2 modulators. In some embodiments, the compounds of the present invention have activity as 5-HT2 modulators. In some embodiments, the compounds of the present invention elicit a biological response by activating a 5-HT2 receptor (e.g., allosteric modulation or modulation of a biological target that activates a 5-HT2 receptor). 5-HT2 agonism has been correlated with the promotion of neural plasticity (Ly et al., 2018). In some embodiments, hallucinogenic potential of a compound of the present invention is determined in vitro. In some embodiments, hallucinogenic potential of a compound of the present invention is determined using a 5-HT2 sensor assay. In some embodiments, the 5-HT2 sensor assay is in an agonist mode or an antagonist mode. In some embodiments, the 5-HT2 sensor assay is in an agonist mode. In some embodiments, a compound of the present invention that does not activate the sensor in agonist mode has non-hallucinogenic potential. In some embodiments, a compound of the present invention that does not activate the sensor in agonist mode is a non-hallucinogenic compound.

In some embodiments, the compounds described herein are 5-HT2 (5-HT_{2A} and/or 5-HT_{2c}) modulators. In some embodiments, the compounds described herein are 5-HT2 modulators and promote neural plasticity (e.g., cortical structural plasticity). In some embodiments, the compounds described herein are 5-HT2 modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT2 modulators (e.g., 5-HT2 agonists) are non-hallucinogenic. In some embodiments, non-hallucinogenic 5-HT2 modulators (e.g., 5-HT2 agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, non-hallucinogenic 5-HT2 modulators (e.g., 5-HT2 agonists) are used to treat neurological diseases. In some embodiments, the neurological diseases comprise decreased neural plasticity, decreased cortical structural plasticity, decreased 5-HT2 receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT2 modulators (e.g., 5-HT2 agonists) are used for increasing neural plasticity. In some embodiments, non-hallucinogenic 5-HT2 modulators (e.g., 5-HT2 agonists) are used for increasing neuronal plasticity. In some embodiments, non-hallucinogenic 5-HT2 modulators (e.g., 5-HT2 agonists) are used for treating a brain disorder. In some embodiments, non-hallucinogenic 5-HT2 modulators (e.g., 5-HT2 agonists) are used for increasing at least one of translation, transcription, or secretion of neurotrophic factors.

### A. Methods for Increasing Neural and Neuronal Plasticity

Neural plasticity refers to the ability of the brain to change structure and/or function throughout a subject's life. Increasing neural plasticity can include increasing neuronal plasticity. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neural plasticity can treat a brain disorder, a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, increasing neuronal plasticity can treat a brain disorder, a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides a method for increasing neural plasticity, comprising contacting a neural cell with a compound of the present invention, or a pharmaceutically acceptable salt thereof, in an amount sufficient to increase neural plasticity of the neuronal cell. In some embodiments, increasing neural plasticity improves a brain disorder described herein.

In some embodiments, the present invention provides a method for increasing neuronal plasticity, comprising contacting a neuronal cell with a compound of the present invention, or a pharmaceutically acceptable salt thereof, in an amount sufficient to increase neuronal plasticity of the neuronal cell. In some embodiments, increasing neuronal plasticity improves a brain disorder described herein.

In some embodiments, the present invention provides a method for increasing neural plasticity, comprising contacting a neural cell with a compound of Formula I:

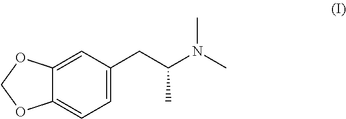

or a pharmaceutically acceptable salt thereof, in an amount sufficient to increase neural plasticity of the neural cell. In some embodiments, the compound has the structure:

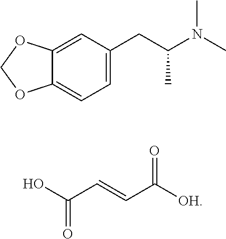

In some embodiments, the present invention provides a method for increasing neuronal plasticity, comprising contacting a neuronal cell with a compound of Formula I:

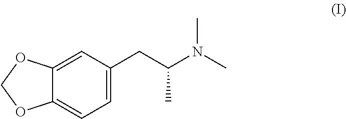

or a pharmaceutically acceptable salt thereof, in an amount sufficient to increase neuronal plasticity of the neuronal cell. In some embodiments, the compound has the structure:

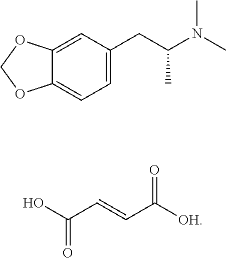

In some embodiments, a compound of the present invention is used to increase neural plasticity. In some embodiments, the compounds used to increase neural plasticity have, for example, anti-addictive properties, antidepressant properties, anxiolytic properties, or a combination thereof. In some embodiments, decreased neural plasticity is associated with a neuropsychiatric disease. In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neural plasticity of any compound of the present invention is a phenotypic assay, a dendritogenesis assay, a spinogenesis assay, a synaptogenesis assay, a Sholl analysis, a concentration-response experiment, a 5-HT2 agonist assay, a 5-HT2 antagonist assay, a 5-HT2 binding assay, or a 5-HT2 blocking experiment (e.g., ketanserin blocking experiments).

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT2 agonist assay, a 5-HT2 antagonist assay, a 5-HT2 binding assay, or a 5-HT2 blocking experiment (e.g., ketanserin blocking experiments). In some embodiments, the experiment or assay to determine the hallucinogenic potential of any compound of Formula I or Formula (Ia) is a mouse head-twitch response (HTR) assay.

In some embodiments, the present invention provides a method for increasing neural plasticity, comprising contacting a neural cell with a compound of the present invention.

In some embodiments, the present invention provides a method for increasing neuronal plasticity, comprising contacting a neuronal cell with a compound of the present invention.

### B. Methods of Treating a Brain Disorder

In some embodiments, the present invention provides a method of treating a disease, including administering to a subject in need thereof, a therapeutically effective amount of a compound of the present invention. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of a compound of the present invention and at least one additional therapeutic agent.

In some embodiments, the present invention provides a method of treating a brain disorder, comprising administering to a subject in need thereof a therapeutically effective amount of a compound of Formula I:

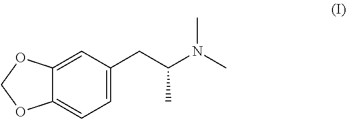

or a pharmaceutically acceptable salt thereof, thereby treating the brain disorder. In some embodiments, the compound has the structure:

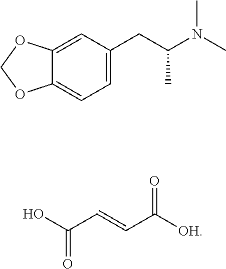

In some embodiments, 5-HT2 modulators (e.g., 5-HT2 agonists) are used to treat a brain disorder. In some embodiments, the brain disorders comprise decreased neural plasticity, decreased cortical structural plasticity, decreased 5-HT2 receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, the brain disorder is one or more of generalized anxiety disorder, phobia, social anxiety disorder, social phobia, panic disorder, panic attack, post-traumatic stress disorders, separation anxiety disorder, selective mutism, agoraphobia, or an anxiety disorder induced by a substance/medication or due to another medical condition. In some embodiments, the methods reduce at least one symptom of anxiety disorder in the subject, such as fear, anxiety, separation anxiety, and/or panic attacks (e.g. frequency and severity of attacks). In some embodiments, the brain disorder is social anxiety disorder. In some embodiments, the brain disorder is post-traumatic stress disorder.

In some embodiments, the present invention provides a method of treating a brain disorder, comprising administering to a subject in need thereof a therapeutically effective amount of a compound of the present invention, or a pharmaceutically acceptable salt thereof, thereby treating the brain disorder.

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder. In some embodiments, the brain disorder is stroke or traumatic brain injury.

In some embodiments, the method further comprises administering one or more additional therapeutic agent that is lithium, Olanzapine (Zyprexa), Quetiapine (Seroquel), Risperidone (Risperdal), Ariprazole (Abilify), Ziprasidone (Geodon), Clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Non-limiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline.

In some embodiments, the present invention provides a compound of the present invention, or a pharmaceutically acceptable salt thereof, or a composition of the present invention, for use in treating a brain disorder.

In some embodiments, the present invention provides use of a compound of the present invention, or a pharmaceutically acceptable salt thereof, or a composition of the present invention, for the manufacture of a medicament for use in treating a brain disorder.

### C. Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neural plasticity, neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increase neural plasticity. In some embodiments, increasing at least one of translation, transcription, or secretion of neurotrophic factors can increase neuronal plasticity. In some embodiments, increasing at least one of translation, transcription, or secretion of neurotrophic factors can include promoting neuronal growth, promoting neuritogenesis, promoting synaptogenesis, promoting dendritogenesis, increasing dendritic arbor complexity, and/or increasing dendritic spine density.

In some embodiments, the present invention provides a method for increasing at least one of translation, transcription or secretion of neurotrophic factors, comprising contacting a neural cell with a compound of the present invention, or a pharmaceutically acceptable salt thereof, in an amount sufficient to increase neural plasticity of the neuronal cell.

In some embodiments, the present invention provides a method for increasing at least one of translation, transcription or secretion of neurotrophic factors, comprising contacting a neuronal cell with a compound of the present invention, or a pharmaceutically acceptable salt thereof, in an amount sufficient to increase neuronal plasticity of the neuronal cell.

In some embodiments, 5-HT2 modulators (e.g., 5-HT2 agonists) are used to increase at least one of translation, transcription, or secretion of neurotrophic factors. In some embodiments, a compound of Formula I or Formula (Ia) described herein is used increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

The neurotrophic factor can be any member of the neurotrophin family such as, but not limited to, Nerve growth factor (NGF), Brain-derived neurotrophic factor (BDNF), neurotrophin 3 (NT-3), neurotrophin 4/5 (NT-4/5), neurotrophin 6 (NT-6), or neurotrophin 7 (NT-7); the neuropoietic cytokines or neurokines such as members of the Ciliary neurotrophic factor (CNTF) family, Leukemia inhibitory factor (LIF), cholinergic differentiation factor, cardiotrophin-1, oncostatin M, growth promoter activity factor, tumor necrosis factor (TNF), interleukin-6 (IL-6), prolactin, growth hormone (GH), leptin, interferon-.alpha., interferon-.beta., and interferon-.gamma.; Epidermal Growth factor (EGF) and Transforming Growth Factor (TGF) families such as of EGF, p185erbB2, p160erbB3, pI80erbB4, neuregulin family including neu differentiation factor or heregulin, acetylcholine receptor-inducing activity, and Glial growth factors (GGFs), TGFalpha, TGFbeta, Glial cell line-derived neurotrophic factor (GDNF), artemin, neurturin, persephin, osteogenic protein-1 (OP-1), bone morphogenetic proteins (BMPs), and growth differentiation factors; Fibroblast growth factor (FGF) family; Insulin-like growth factor (IGF) family; platelet-derived growth factor (PDGF) family; Hepatocyte growth factor (HGF) family; granulocyte-colony stimulating factor (G-CSF) family; neuroimmunophilins; pigment epithelium-derived factor (PEDF) family; activity-dependent neurotrophic factors such as activity-dependent neuroprotective protein (ADNP) and neuritin (activity-induced neurotrophic factor); angiogenesis growth factor family; vascular endothelial growth factor (VEGF) family; cerebral dopamine neurotrophic factor (CDNF) family; mesencephalic astrocyte-derived neurotrophic factor (MANF) family; Ephrins family such as ephrin A1, ephrin A2, ephrin A3, ephrin A4, ephrin A5, ephrin B1, ephrin B2, and ephrin B3; DHEA; glia maturation factor; insulin; pituitary adenylate cyclase-activating peptide (PACAP); interleukin-1 (IL-1); interleukin-2 (IL-2); interleukin-3 (IL-3); interleukin-5 (IL-5); interleukin-8 (IL-8); macrophage colony-stimulating factor (M-CSF); granulocyte-macrophage colony-stimulating factor (GM-CSF); neurotactin; neurotransmitters and neuromodulators; serine protease inhibitors such as protease nexin-1, hedgehog family of inducing proteins; proteins involved in synapse formation such as agrin, laminin 2, and ARIA (ACh-inducing activity); variants and combinations thereof. In some embodiments, the neurotrophin factors can be Nerve growth factor (NGF), Brain-derived neurotrophic factor (BDNF), neurotrophin 3 (NT-3), neurotrophin 4/5 (NT-4/5), neurotrophin 6 (NT-6), neurotrophin 7 (NT-7), variants and combination thereof.

In some embodiments, the present invention provides a method for increasing at least one of translation, transcription or secretion of neurotrophic factors, comprising contacting a neural cell with a compound of Formula I:

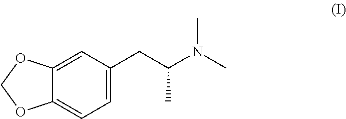

or a pharmaceutically acceptable salt thereof, in an amount sufficient to increase neural plasticity of the neural cell. In some embodiments, the compound has the structure:

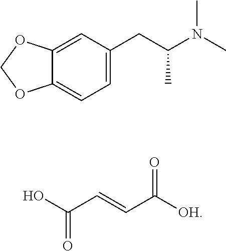

In some embodiments, the present invention provides a method for increasing at least one of translation, transcription or secretion of neurotrophic factors, comprising contacting a neuronal cell with a compound of Formula I:

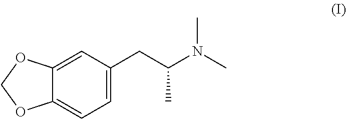

or a pharmaceutically acceptable salt thereof, in an amount sufficient to increase neuronal plasticity of the neuronal cell. In some embodiments, the compound has the structure:

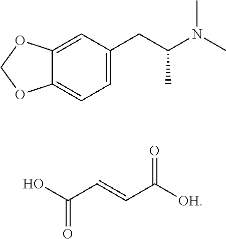

### D. Methods for Modulating a 5-HT2 Receptor

In some embodiments, the present invention provides a method for modulating a 5-HT2 receptor, comprising contacting a neural cell with a compound of the present invention, or a pharmaceutically acceptable salt thereof, or a composition of the present invention, in an amount sufficient to modulate the 5-HT2 receptor.

In some embodiments, the present invention provides a method for modulating a 5-HT2 receptor, comprising contacting a neuronal cell with a compound of the present invention, or a pharmaceutically acceptable salt thereof, or a composition of the present invention, in an amount sufficient to modulate the 5-HT2 receptor.

Modulating the 5-HT2 receptor can include agonising or antagonizing the 5-HT2 receptor. In some embodiments, the present invention provides a method for agonising a 5-HT2 receptor, comprising contacting a neural cell with a compound of the present invention, or a pharmaceutically acceptable salt thereof, or a composition of the present invention, in an amount sufficient to agonise the 5-HT2 receptor. In some embodiments, the present invention provides a method for agonising a 5-HT2 receptor, comprising contacting a neuronal cell with a compound of the present invention, or a pharmaceutically acceptable salt thereof, or a composition of the present invention, in an amount sufficient to agonise the 5-HT2 receptor. In some embodiments, the present invention provides a method for antagonizing a 5-HT2 receptor, comprising contacting a neural cell with a compound of the present invention, or a pharmaceutically acceptable salt thereof, or a composition of the present invention, in an amount sufficient to antagonize the 5-HT2 receptor. In some embodiments, the present invention provides a method for antagonizing a 5-HT2 receptor, comprising contacting a neuronal cell with a compound of the present invention, or a pharmaceutically acceptable salt thereof, or a composition of the present invention, in an amount sufficient to antagonize the 5-HT2 receptor.

The 5-HT2 receptor includes, but is not limited to, the 5-HT2A, 5-HT2B, or 5-HT2C receptor. In some embodiments, The 5-HT2 receptor is at least one of 5-HT2A, 5-HT2B, or 5-HT2C receptor. In some embodiments, the 5-HT2 receptor is 5-HT2A. In some embodiments, the 5-HT2 receptor is 5-HT2B. In some embodiments, the 5-HT2 receptor is 5-HT2C.

In some embodiments, the present invention provides a method for treating a disease or condition responsive to the modulation of a 5-HT2 receptor, comprising contacting a neural cell with a compound of the present invention, or a pharmaceutically acceptable salt thereof, or a composition of the present invention, in an amount sufficient to treat the disease or condition responsive to modulation of the 5-HT2 receptor. In some embodiments, the present invention provides a method for treating a disease or condition responsive to the modulation of a 5-HT2 receptor, comprising contacting a neuronal cell with a compound of the present invention, or a pharmaceutically acceptable salt thereof, or a composition of the present invention, in an amount sufficient to treat the disease or condition responsive to modulation of the 5-HT2 receptor. The 5-HT2 receptor includes, but is not limited to, the 5-HT2A, 5-HT2B, or 5-HT2C receptor. In some embodiments, The 5-HT2 receptor is at least one of 5-HT2A, 5-HT2B, or 5-HT2C receptor. In some embodiments, the 5-HT2 receptor is 5-HT2A. In some embodiments, the 5-HT2 receptor is 5-HT2B. In some embodiments, the 5-HT2 receptor is 5-HT2C.

### VI. Examples

Chemistry (General). All reagents were obtained commercially unless otherwise noted. Reactions were performed using glassware that was oven dried (120° C.) unless otherwise stated. Air- and moisture-sensitive liquids and solutions were transferred via syringe or stainless-steel cannula. Organic solutions were concentrated under reduced pressure (˜5 Torr) by rotary evaporation. Solvents were purified by passage under 12 psi N₂ through activated alumina columns. Chromatography was performed using Fisher Chemical™ Silica Gel Sorbent (230-400 Mesh, Grade 60). Compounds purified by chromatography were typically applied to the adsorbent bed using the indicated solvent conditions with a minimum amount of added dichloromethane as needed for solubility. Thin layer chromatography (TLC) was performed on Merck silica gel 60 F254 plates (250 μm). Visualization of the developed chromatogram was accomplished by fluorescence quenching or by staining with butanolic ninhydrin, aqueous potassium permanganate, ethanolic vanillin, or aqueous ceric ammonium molybdate (CAM).

Nuclear magnetic resonance (NMR) spectra were acquired on either a Bruker 400 operating at 400 and 100 MHz or a Varian-600 operating at 600 and 150 MHz for ¹H and ¹³C, respectively, and are referenced internally according to residual solvent signals. Data for 1H NMR are recorded as follows: chemical shift (6, ppm), multiplicity (s, singlet; br s, broad singlet; d, doublet; t, triplet; q, quartet; quint, quintet; sext, sextet; m, multiplet), integration, and coupling constant (Hz). Data for ¹³C NMR are reported in terms of chemical shift (6, ppm). Infrared spectra were recorded using a Thermo Scientific Nicolet iS10 spectrometer with Smart iTX Accessory (diamond ATR) and are reported in frequency of absorption. Low-resolution mass spectra were obtained using a Waters Acuity Arc LC-MS.

The specific procedures used to synthesize the compounds reported in this manuscript are detailed below along with characterization data. Spectral data (¹H and ¹³C NMR spectra) for each compound tested in biological assays is included.

### General

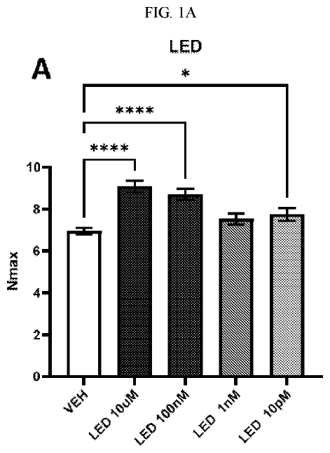

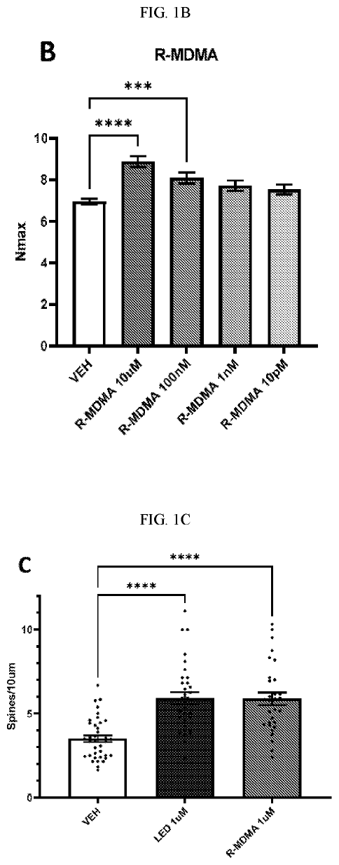

LED and L-MDMA Promote Neural Plasticity. Our previous research has demonstrated that classical psychedelics and their analogs are capable of robustly promoting neural plasticity in cortical neurons. This is believed to underly many of their therapeutic properties in regards to treating mood and anxiety disorders where a common underlying observation is the atrophy and dysregulation of cortical neurons and their respective circuits. While MDMA is not a classical psychedelic, it has been demonstrated that it promotes plasticity in cortical cultures, presumably through a non-efflux mechanism as SERT isn't expressed in these neurons. This effect is likely to be critical to its potential long-lasting therapeutic effects and ability to induce changes in damaged cortical circuits. To examine the effects of R-MDMA and LED in promoting neural plasticity cultured embryonic rat cortical neurons were treated with increasing concentrations of each compound. Using Sholl analysis, it was observed that both robustly increased dendritic arbor complexity, possessing efficacy in the nanomolar range (FIG. 1A,B). In addition to promoting dendritic growth, LED also increases dendritic spine density, comparably to R-MDMA, in mature cortical cultures (FIG. 1C).

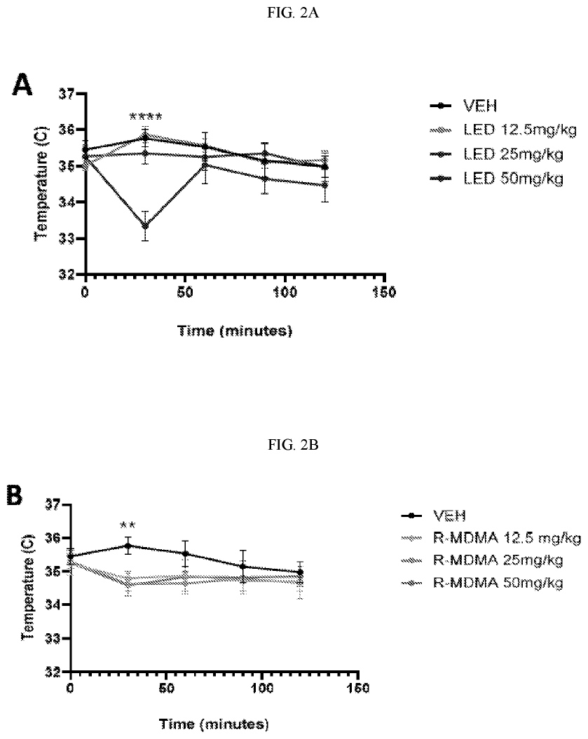

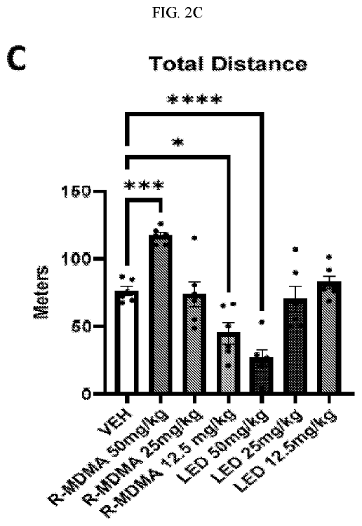

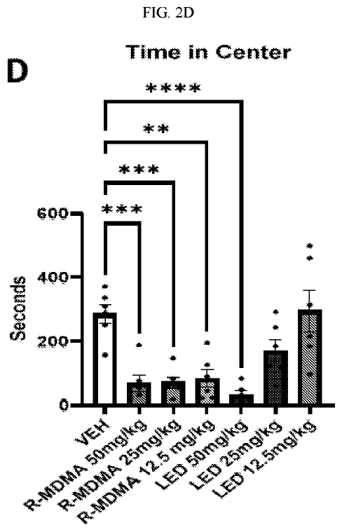

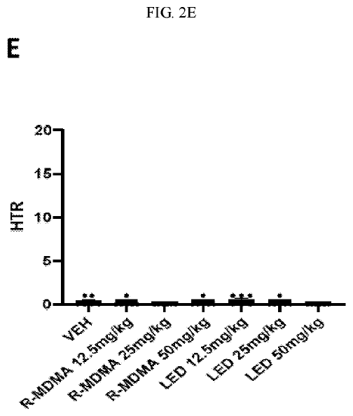

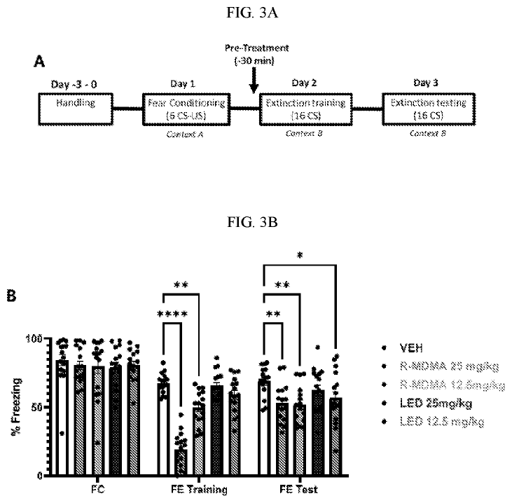

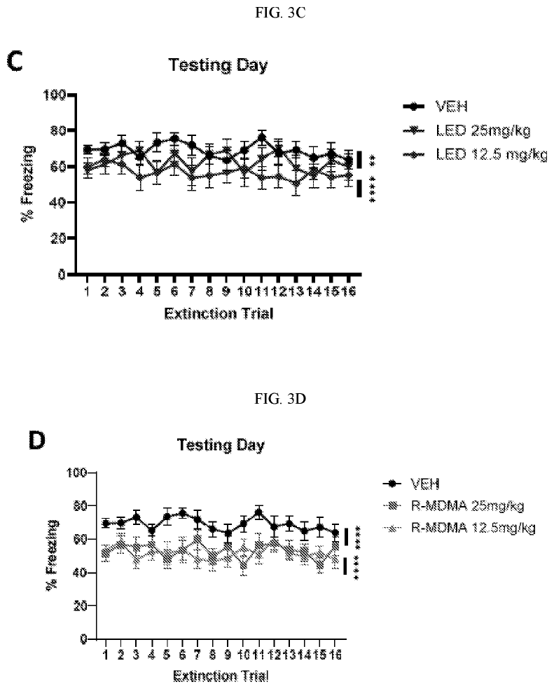

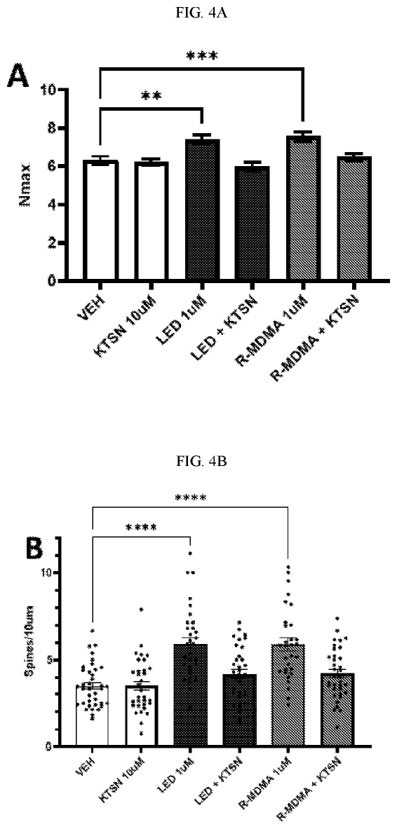

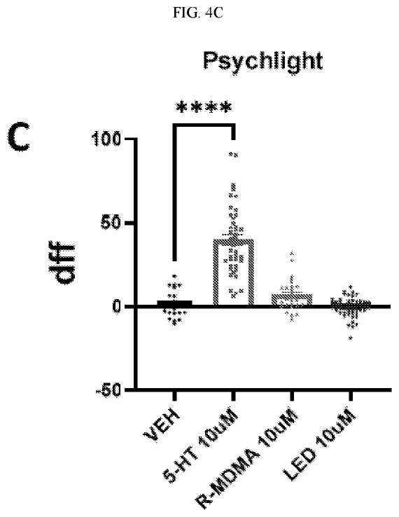

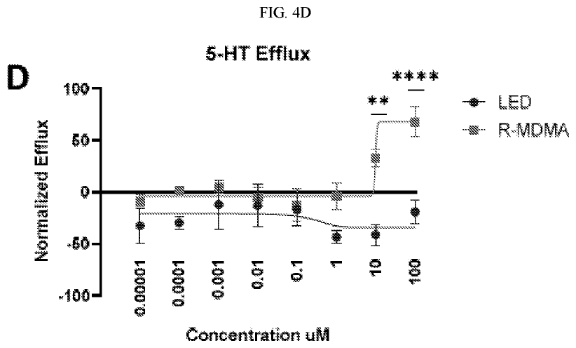

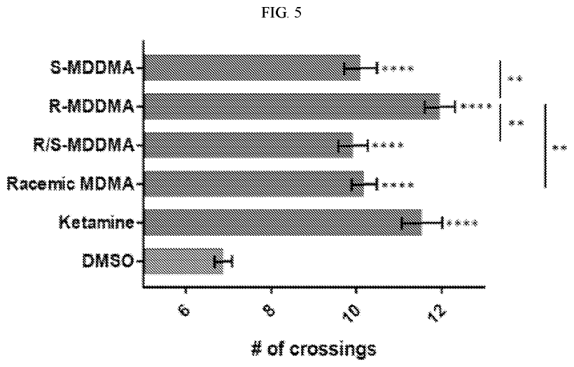

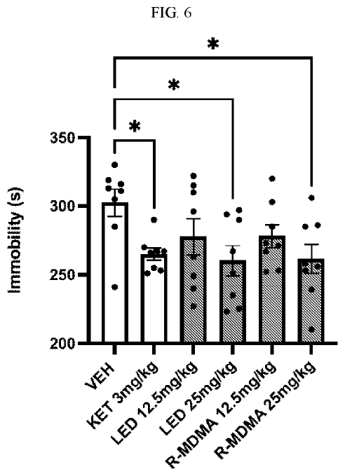

LED and L-MDMA Safety Profiles. One of the biggest draw backs and concerns with MDMA as a therapeutic is it's abuse potential stemming from its structural similarity to amphetamines, a potent class of dopamine releasers. While MDMA is more selective for SERT compared to the dopamine transporter (DAT), especially when using the R enantiomer—previous studies have demonstrated that at doses of 10 mg/kg or lower there is minimal dopamine release—it is still capable of causing dopamine release within the midbrain and thus possess inherent abuse potential. The dopamine releasing properties of drugs of abuse such as amphetamine result in a strong psychostimulant effect that can be measured via increases in locomotion. To this end a novelty induced locomotion (NIL) test was performed where mice were treated with either R-MDMA or LED and immediately placed in novel arena. We chose three doses (12.5 mg/kg, 25 mg/kg, and 50 mg/kg) of both compounds with the middle dose of 25 mg/kg based on previous studies by Howell and coworkers that showed 17 mg/kg of R-MDMA displays therapeutic properties without psychostimulant and hyperthermic properties of RS-MDMA. We observed that R-MDMA causes a dose-dependent increase in locomotion where the highest dose of 50 mg/kg exhibits psychostimulant effects (FIG. 2C). This is contrasted with LED's dose dependent decrease in locomotion where the 50 mg/kg dose causes a significant decrease in locomotion compared to the vehicle control (FIG. 2C). This is a striking finding, displaying the divergence of effects and underlying pharmacology between the two compounds. Furthermore, this is the first evidence that R-MDMA exhibits psychostimulant effects, suggesting that there may still be a dopamine releasing component to the compound and thus abuse potential if used at sufficiently high doses. This is contrasted with LED which fails to display any psychostimulant properties and suggests that the chemical modification has resulted in substantial decrease in abuse potential. We also measured time spent in the center of the arena to assess whether either compound had anxiolytic or anxiogenic effects. All doses of R-MDMA induced a large decrease in time spent in the center, suggesting there may be an anxiogenic component to these doses of R-MDMA (FIG. 2D). Previous studies using the elevated plus maze have shown that RS-MDMA has a complex effect on anxiety like behavior where low acute and subchronic doses tend to be anxiogenic but higher acute and subchronic doses produce anxiolytic effects in the EPM. This is again contrasted with LED where only the highest dose caused a decrease in time spent in the center (FIG. 2D).

In addition to abuse potential SR-MDMA is known to be capable of inducing severe hyperthermia at high doses and is typically correlated with potential neurotoxicity characterized by reactive astrogliosis and neuronal terminal pruning. To test for a propensity for either compound to induce increases in body temperature, body temperature of mice were recorded every 30-minutes after being treated with R-MDMA or LED for 2-hours using an IR thermometer. Neither compound induced any increases in body temperature, in fact both R-MDMA and LED caused a decrease in body temperature compared to the vehicle control at the 30-minute time point, suggesting that neither compound is likely to induce hyperthermia at these doses (FIG. 2A,B). At higher doses RS-MDMA has been described to be weakly hallucinogenic producing simple visual distortions described as flashes of light in the peripheral visual field. To test for the possibility of any hallucinogenic potential in either compound, recordings of mice while in the novelty induced arena were assessed and failed to demonstrate any HTRs that would suggest either compound possess hallucinogenic potential (FIG. 2E). The combination of these tests demonstrates that LED has an improved safety profile compared to R-MDMA which possess psychostimulant properties when used at higher doses, and also suggests a fundamental divergence of pharmacological mechanism based on the opposite effects on locomotion.

LED and R-MDMA Promote Fear Extinction. Both RS-MDMA and R-MDMA are known to promote fear extinction in rodents and are believed to underly the therapeutic effects of MDMA in treating PTSD, a condition believed to be caused by impairments in the extinction of fear memories. Extinction involves a learning process where the fear response attenuates to a point it is no longer a reliable predictor of the expected threat. RS-MDMA administered systemically, to the basolateral amygdala (BLA), or infralimbic cortex (IL) prior to extinction training is sufficient to induce lasting improvements in fear memory extinction through a SERT, 5-HT2A, and BDNF dependent mechanism. To directly compare the effects of R-MDMA to LED Pavlovian-cued fear conditioning was performed followed by a fear extinction paradigm using the 12.5 mg/kg and 25 mg/kg doses as neither of them altered changes in locomotion or had psychostimulant properties (FIG. 2A). Both doses of R-MDMA given 30 minutes before extinction training caused substantial reductions in freezing to the CS during the training session (FIG. 2B). Twenty-four hours later, tested in the extinction context in a drug-free state, mice previously given R-MDMA before extinction training exhibited similar reductions of conditioned freezing (FIG. 2B,D). The reduction in freezing caused by R-MDMA during extinction training can't be attributed to changes in locomotion as neither dose had an effect in the NIL test. LED again displayed some divergent properties as neither dose influenced freezing during extinction training but the 12.5 mg/kg dose caused a significant reduction in freezing during testing day 24-hours later (FIG. 2B,C). This result can be further broken down as both females and males were used in this experiment; when separated by sex it appears that there is a sex specific effect where the males display enhanced extinction with the 25 mg/kg dose and the females display enhanced extinction with the 12.5 mg/kg dose, suggesting females may be more sensitive to the effects of LED (SI). When combined together the females carry the response to the 12.5 mg/kg dose and appear to also be more sensitive to the effects of R-MDMA compared to males. This result highlights the utility of LED as both a potential safer alternative to R-MDMA as it can promote fear extinction without possessing any psychostimulant properties, and as a chemical tool to study the mechanism of action of MDMA.

Mechanism of Action. The mechanism of action for MDMA is generally believed to be mediated via actions at monoamine transporters, with SERT being the primary transporter. MDMA acts as a substrate for monoamine transporters which leads to some reuptake inhibition though it is a more potent releaser of monoamines via its actions on the transporters, vesicular amine transporters (VMAT), and its weak base properties. The increases in synaptic serotonin, norepinephrine, and dopamine lead to increased activity at post synaptic receptors and concomitant downstream signaling believed to mediate the majority of MDMA's behavioral effects. In addition, there is evidence that MDMA may bind directly to a host of different receptors including serotonin, adrenergic, muscarinic, dopamine, and trace-amine associated receptors. These interactions are understudied when compared to MDMAs effects on monoamine transporters but may play an important role in its pharmacology. We chose to use LED, with its structure almost identical as MDMA, as a chemical tool to compare and contrast mechanisms of action. To begin it was assessed how the small chemical modification of LED would alter it's ability promote [³H]5-HT release using HEK293T cells transiently expressing SERT. As these cells lack substrate-filled vesicles, SERT is the sole carrier of released [³H]5-HT. We observed that LED failed to induce [³H]5-HT release at all concentrations tested (10 pM-100 uM), this is contrasted with R-MDMA where the two highest concentration of 10 uM and 100 uM caused significant release of [³H]5-HT when compared to LED. The high concentration of R-MDMA needed to cause [³H]5-HT release is not surprising as it is known the R enantiomer is a weak monoamine releaser. Other studies have demonstrated that the racemic version of LED has a moderate ability to inhibit uptake of 5-HT as well as promote its release, suggesting that this effect is primarily mediated by the D enantiomer, as is the case with MDMA. This result is striking as this suggests LED lacks the primary mechanism through which MDMA is believed to exert its behavior effects, yet it still promotes extinction learning.

Next, it was tested whether the ability for R-MDMA and LED to promote plasticity as measured via dendritic arborization and dendritic spine density are dependent on 5-HT2 signaling. To this end cortical cultures with ketanserin, a 5-HT2 antagonist, were pre-treated followed by treatment with R-MDMA or LED; both compounds ability to promote plasticity are inhibited by the antagonist, suggesting that 5-HT2 signaling is involved in the plasticity promoting effects for both compounds. Given that there are no serotonergic afferents in the cultures for MDMA to cause release of serotonin and subsequent activation of 5-HT2 receptors, this suggests that the plasticity promoting effects of MDMA and LED are mediated via direct binding to 5-HT2 receptors or indirectly causing activation of 5-HT2 receptors.

To examine potential interactions with 5-HT receptors Psychlight, a 5-HT2A biosensor was expressed in cultured cortical neurons. The results from the 5-HT2A biosensor demonstrate that neither R-MDMA nor LED, when dosed at 10 uM, induce a statistically significant change in the sensor fluorescence when compared to the vehicle control; though it appears that R-MDMA activates the sensor to a greater degree than LED, suggesting there may be some weak activity, or a higher dose is needed to activate the sensor. Because ketanserin also is a high affinity inhibitor of 5-HT2C the possibility exists that R-MDMA and LED may be acting through agonism of the 5-HT2C receptor, and it is planned to test this with the use of a 5-HT2C biosensor. As it stands the difference between R-MDMA and LED mechanism(s) of action is the ability to promote 5-HT release; this mechanism, believed to be critical for the prosocial effects of RS-MDMA, is preserved in R-MDMA but lost in LED. This suggests that LED is likely working via direct or indirect agonism of receptors, potentially the 5-HT2C receptor.

Drugs. For cell culture experiments, the vehicle was a 0.1% (agonist studies) or 0.2% (antagonist studies) molecular biology grade dimethyl sulfoxide (DMSO) (Sigma-Aldrich) solution. For in vivo experiments, the vehicle was USP grade saline (0.9%). The fumarate salt of R-3,4-Methylenedioxymethamphetamine (2:1, L-MDMA:fumaric acid) and R-3,4-Methylenedioxy-N, N-dimethylamphetamine (1:1, L-MDMA:fumaric acid) were synthesized in-house, as described previously, and judged to be analytically pure on the basis of nuclear magnetic resonance (NMR) and liquid chromatography-mass spectrometry (LC-MS) data. Other chemicals were purchased from commercial sources as follows: ketamine hydrochloride (Fagron), ketanserin (ApexBio), 5-[1,2-H[N]]-hydroxytryptamine creatinine sulfate (Perkinelmer), and DL-p-chloroamphetarmine (Sigma Aldrich).

Animals. All experimental procedures involving animals were approved by the University of California, Davis Institutional Animal Care and Use Committee (IACUC) and adhered to the principles described in the NIH Guide for the Care and Use of Laboratory Animals. E18 timed pregnancy Sprague-Dawley rats were obtained from Charles River Laboratories (Wilmington, MA, USA). C57BL/6J mice were obtained from Jackson Laboratory and were approximately 9-13 weeks old at the time of the experiments.

### Example 1. Dendritogenesis Experiments

For the dendritogenesis experiments conducted using cultured cortical neurons, E18 timed pregnant Sprague Dawley rats were obtained from Charles River Laboratories. Full culturing, staining and analysis experiments were performed as previously described with the exception that there was no media change during treatments. Dendrites were visualized using a chicken anti-MAP2 antibody (1:10,000; EnCor, CPCA-MAP2) as previously reported. DMSO and ketamine (10 pM) were used as vehicle and positive controls, respectively. For blocking experiments, ketanserin was added 30-minutes prior to addition of agonists in 10-fold excess.

### Example 2. Spinogenesis Experiments

Spinogenesis experiments were performed as previously described with the exception that cells were treated at DIV14 instead of DIV18 and then fixed on DIV15. The images were taken on a Nikon HCA Confocal microscope a with a 100x/NA 1.45 oil objective. DMSO and ketamine (10 pM) were used as vehicle and positive controls, respectively. For blocking experiments, ketanserin was added 30-minutes prior to addition of agonists in 10-fold excess.

Statistical Analysis. Appropriate samples sizes were estimated based on our previous experiences performing similar experiments. Treatments were randomized, and data were analyzed by experimenters blinded to treatment conditions. All comparisons were planned prior to performing each experiment. Statistical analyses were performed using GraphPad Prism (v.8.1.2). Data are represented as mean±s.e.m., unless otherwise noted, with asterisks indicating *P<0.05, **P<0.01, ***P<0.001 and ****P<0.0001.

### Example 3. Head-Twitch Response, Novelty Induced Locomotion, and Body Temperature Assay

The head-twitch response assay was performed as described previously using both male and female C57BL/6J mice (3 each per treatment). The mice were obtained from Jackson Laboratory and were approximately 9 weeks old at the time of the experiments. Before injecting the mice, their baseline temperature was measured using an IR thermometer as described. Compounds were administered via intraperitoneal (i.p.) injection (5 ml kg-1) using 0.9% saline as the vehicle. Immediately after the animals were placed in an arena for 30-minutes where their locomotor activity was assessed using AnyMaze automated tracking software. The behavior was also videotaped and later scored by two blind overserves for head-twitch responses, the results were averaged. Upon completion of the novelty induced locomotion assay their temperature was measured at the 30-minute mark. The mice were then transferred to a holding cage with their other treatment matched cage mates and their temperature was measured at the 90 and 120-minute mark.

### Example 4. Fear Extinction

Mice were exposed to cued fear conditioning on day 1, fear extinction training on day 2 and extinction testing on Day 3. Mice were habituated to handling for 3 days before experimentation. On the day of the experiment mice were allowed to habituate to the behavior room for 1-hour before beginning the experiment. 15-minutes before starting the experiment, mice were separated out into individual holding cages.

Cued fear conditioning consisted of six pairings of a CS auditory cue (80-85 dB white noise, 15 s) and a co-terminating US footshock (0.85 mA and 2 s), each spaced 1.5 minutes apart. The fear conditioning apparatus (Med Associates model #MED-VFC2-SCT-R) consisted of a 30.5 cm×24.1 cm×21 cm internal soundproof chamber, with metal grated floors, an infrared camera, a sound generator, and a light source. Mice were initially placed in the conditioning apparatus and the first 1.5 minutes were stimulus free. After the last shock, the animals remained in the chambers for an additional 2 min before being returned to their home cages. During fear conditioning, the apparatus was illuminated to 100 lx and did not contain any additional odor cues. Freezing responses for fear conditioning (day 1) are presented as the percentage of time spent freezing during the last 1.5 minutes after the last shock. The apparatus was cleaned with 70% EtOH in between trials.

On day 2, animals were separated into individual holding cages and administered either vehicle (0.9% USP saline), R-MDMA (12.5 or 25 mg/kg IP) or R-MDDMA (12.5 or 25 mg/kg I.P.) 30-minutes prior being placed in the fear extinction context. Cued fear memory was assessed by exposing the animals to a novel context (lights off, A-frame insert, smooth plastic floor insert, additional vanilla odor) for 2 min prior to 16 presentations of auditory cues (80 dB white noise, 15 s) spaced 15 s apart. Freezing responses for cue testing (day 2 and 3) are presented as the percentage of time spent freezing during the tone presentations. The procedure was repeated on day 3 in the absence of drug.

Fear conditioning experiments were performed between the hours of 08:00-11:00. Freezing behavior was scored using Med Associates Video Freeze software v2.25 (motion threshold=100 au, detection method=linear, minimum freeze duration=15 frames, which is equal to a 0.5 s freeze). The apparatus was cleaned with 70% EtOH in between trials on day 1 and Sani-cloth gemicidal disposable cloths (PDI, Q89072) were used to clean the apparatus on days 2 and 3.

### Example 5. SERT Efflux Experiments

HEK293T cells were grown in Dulbecco's Modified Eagle Media (DMEM) supplemented with 10% fetal bovine serum (FBS) and 1% penicillin-streptomycin. 24 hours prior to the experiment, cells were plated in 24-well plates at a density of 150,000 cells/well and concurrently transfected using Lipofectamine 3000 according to the manufacturer's protocol. Cells were transfected with hSERT-N1-pEYFP (Addgene #70105) or a GFP control plasmid (Lonza pmaxGFP vector). The next day the wells were washed (1×500 μL) with 1× Hank's Balanced Salt Solution supplemented with 2 mM MgCl₂ and 2 mM CaCl₂ (HBSS) and the plate was placed on a 37° C. water bath for the remainder of the experiment. 250 μL of HBSS containing 10 nM 5-[1,2-3H[N]]-hydroxytryptamine creatinine sulfate (NET498001MC, Lot: 2902700) was added to the wells and incubated for 15 minutes, allowing uptake into SERT expressing HEK cells. Uptake was terminated by aspiration of wells followed by washes (3×500 μL) with HBSS. Efflux was initiated by adding 250 μL drug solution (0.02% DMSO as vehicle control, 100 μM DL-p-chloroamphetamine (Sigma Aldrich, C9635-1G) as the positive control, 100 μM-10 μM L-MDMA, 100 μM-10 μM L-MDDMA) in HBSS for 15 minutes. 200 μL of media was collected from each well and placed into a scintillation vial containing 1 mL PerkinElmer Ultima Gold™ scintillation cocktail to serve as the efflux fraction. The wells were washed (3×500 μL) with HBSS and then cells were lysed for 10 minutes by adding 250 μL radioimmunoprecipitation assay (RIPA) buffer (50 mM Tris HCl, 150 mM NaCl, 1% NP-40, 0.5% sodium deoxycholate, 0.1% SDS, pH 8) to each well. Cells were lysed with a P1000 pipette using the RIPA buffer in the well and all contents of the well were collected into separate scintillation vials containing 1 mL PerkinElmer Ultima Gold™ scintillation cocktail to serve as the intracellular fraction. An additional 250 μL of RIPA buffer was added to each well and then transferred to the intracellular fraction vials. Scintillation vials were mixed by inverting incubated overnight at room temperature. Radioactivity in the form of disintegrations per minute (DPM) was determined the following day with the use of a Beckman LS 6000 liquid scintillation counter. The efflux fraction counts were corrected by a factor of 1.25 to account for the 50 μL remaining in each well (DPM*250 μL total/200 μL collected). Using the DPM for each compound, bar graphs were reported as efflux fraction/(efflux fraction+intracellular fraction) and then normalized to the vehicle control and positive control with the vehicle set at 0% and positive control set at 100%. The GFP control cells were checked to ensure radioactivity was below background levels.

### Example 6. Sensor Experiments

E18 rat cortical neurons were plated at 60,000 cells/well in glass 96-well plates (P96-1.5H-N, Cellvis) coated with poly-D-lysine (Sigma, P6407-5MG). At day in vitro (DIV) 3-7 neurons were infected with AAV9_hSynapsin_psychLight2 (Neurophotonics-Viral Vector Core) or AAV9-hsyn-GRAB_5-HT1.0 (Addgene, 140552-AAV9) at an MOI of 70,000. Neurons were kept in the incubator for one week to allow for viral expression. On imaging day (DIV 10-14), media was aspirated and wells were washed (1×200 μL) with HBSS supplemented with 2 mM MgCl₂ and 2 mM CaCl₂. 50 μL HBSS was added to the wells of the assay plate and then imaged on a CellInsight CX7 HCA Platform (Thermo Fisher, CX7A1110) configured with a onstage incubator for live cell imaging (Thermo Fisher, NX7LIVE001) at 40×(N.A.=0.60) with 25 regions of interest (ROI) taken per well using an arbitrary ROI pattern for each well with no bias to location and no overlap of the ROIs (exposure=400 ms, LED power=100%). Immediately prior to imaging, stock solutions of drugs in DMSO (10 mM) were diluted 1:500 in imaging media (HBSS) and distributed across an empty 96-well treatment plate in triplicate following a randomized plate map. Next, 50 μL from the treatment plate was transferred to the assay plate resulting in a 1:1000 dilution of drug (10 μM as the final concentration in 0.1% DMSO). As a positive and vehicle control, 5-HT (10 μM) and 0.1% DMSO were used, respectively. After a 5-minute incubation, the same sites were re-imaged using the same settings.

Images were then analyzed using ImageJ by selecting ROIs surrounding individual neuronal cell bodies of the post-treatment images and recording the mean fluorescence, the same ROIs were applied to the pretreatment images to record the mean fluorescence. The AF/F values were calculated as previously reported.

### Example 7. Synthesis of R-MDDMA Fumarate

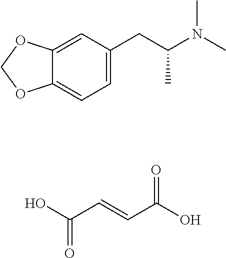

Tert-butyl (R)-(1-hydroxypropan-2-yl)carbamate. To an ice-cold solution of (2R)-2-aminopropan-1-ol (6.7 g, 90.0 mmol) in DCM (180 mL) was added triethylamine (25.2 mL, 180 mmol, 2 equiv.) and Di-tert-butyl dicarbonate (23.5 g, 108.0 mmol, 1.2 equiv.) portion-wise. The reaction was brought to room temperature and stirred overnight before being diluted with DCM (100 mL) and H₂O (300 mL). The phases were separated, and the aqueous phase was extracted with DCM (2×100 mL). The organic extracts were combined, dried over Na₂SO₄, filtered, concentrated under reduced pressure, and purified via flash chromatography (7:3 Hexanes/EtOAc). The resulting white solid was dried under reduced pressure to yield the pure compound. Yield=11.0 g, 70%.

Tert-butyl (R)-2-methylaziridine-1-carboxylate. To an ice-cold solution of Tert-butyl (R)-(1-hydroxypropan-2-yl)carbamate (1.0 g, 5.7 mmol) in diethyl ether (114 mL) was added p-toluenesulfonyl chloride (1.6 g, 8.6 mmol, 1.5 equiv.) then freshly powdered potassium hydroxide (1.3 g, 23.0 mmol, 4.0 equiv.) portion-wise. The reaction was brought to room temperature and stirred 6 h before being filtered, washing with an ice cold portion of diethyl ether (30 mL). The filtrate was concentrated under reduced pressure and used without further purification. Yield=514 mg, 57%.

tert-butyl (R)-(1-(benzo[d][1,3]dioxol-5-yl)propan-2-yl)carbamate. To a −78° C. solution of copper(I) bromide dimethyl sulfide (25 mg, 0.12 mmol, 0.3 equiv.) in THF (2.3 mL) was added 3,4-(Methylenedioxy)phenylmagnesium bromide solution(0.8M in toluene:THF 50:50) (1.1 mL, 0.9 mmol, 2.2 equiv.). After 15 min, a solution of tert-butyl (R)-2-methylaziridine-1-carboxylate (65 mg, 0.41 mmol) in THF (2 mL) was added drop-wise. The reaction was warmed to room temperature slowly and stirred 1 h before being diluted with a sat'd solution NH₄Cl (30 mL) and EtOAc (15 mL). The phases were and the aqueous phase was extracted with EtOAc (3×15 mL). The organic extracts were combined, dried over Na₂SO₄, filtered, concentrated under reduced pressure, and purified via flash chromatography (9:1 Hexanes/EtOAc). The resulting tan solid was dried under reduced pressure to yield the pure compound. Yield=60 mg, 52%. [0144] (R)-1-(benzo[d][1,3]dioxol-5-yl)-N,N-dimethylpropan-2-amine (R-MDDMA Fumarate 1:1). To an ice-cold solution of tert-butyl (R)-(1-(benzo[d][1,3]dioxol-5-yl)propan-2-yl)carbamate (230 mg, 0.82 mmol) in CHCl₃ (2 mL) was added trifluoroacetic acid (0.5 mL) and stirred for 15 min. The reaction was diluted with MeOH (5 mL) and concentrated under reduced pressure. The crude material was taken up in MeOH (21 mL) and cooled to 0° C. Acetic acid (0.24 mL, 4.2 mmol, 5 equiv.) was added to the reaction mixture followed by NaBH₃CN 110 mg, 1.75 mmol, 2.1 equiv.) and the reaction was warmed to room temp. The reaction was stirred overnight before being concentrated under reduced pressure. The resulting crude solid was taken up in 1 M NaOH_{(aq)} (100 mL) and DCM (25 mL). The phases were and the aqueous phase was extracted with DCM (3×25 mL). The organic extracts were combined, dried over Na₂SO₄, filtered, and concentrated under reduced pressure. The resulting solid was taken up in acetone (1 mL) and added to a boiling solution of fumaric acid (97 mg, 0.84 mmol, 1 equiv.) in acetone (18 mL). The solution was cooled to room temperature and stored at −20° C. overnight. The solid was filtered and washed with ice cold acetone (1 mL) and dried under reduced pressure to afford the desired product as the 1:1 fumarate salt. Yield=194 mg, 72%. 1H NMR (600 MHz, CD₃OD) δ 6.80 (m, 2H), 6.74 (t, 1H, J=8.08 Hz), 6.70 (s, 2H), 5.94 (s, 2H), 3.6 (m, 1H), 3.08 (t, 1H, J=4.34, 8.71 Hz), 2.87 (s, 6H), 2.71 (m, 1H), 1.21 (d, 3H, J=6.71 Hz), ppm; 13C NMR (150 MHz, CD_{3OD)} δ 170.41, 149.56, 148.33, 135.90, 130.89, 123.61, 110.41, 109.45, 102.47, 64.46, 39.62, 37.79, 12.62, ppm. LRMS (ES+) m/z caled for C₁₆H₂₁NO₆+207.13, found 208.34 (MH+).

### Example 8. Tail Suspension Test

Male C57/BL6J mice (9-10 weeks of age at time of experiment) were obtained from the Jackson Lab and housed in a UCD vivarium. After 1 week of habituation to the vivarium each mouse was handled for approximately 1 minute by a male experimenter for three consecutive days, and on the fourth day mice were administered VEH (saline), KET 3 mg/kg (ketamine), LED 12.5 or 15 mg/kg, and R-MDMA 12.5 or 25 mg/kg via IP injection (5 mL/kg). All experiments were carried out by the same experimenter who performed the handling. Twenty-four hours after treatments, animals were subjected to a tail suspension test. Animals were suspended from their tails approximately 16 cm from the top of the apparatus and 25 cm from the bottom of the apparatus. Behavior was video recorded for 6 min and analyzed later by an experimenter blinded to treatment conditions. Immobility was defined as the amount of time spent not struggling or attempting to generate momentum and was scored for the entire 6 min period. All TST sessions were performed between the hours of 08:00 and 13:00.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula: 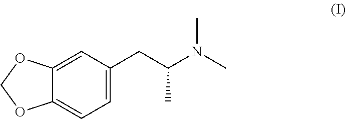
or a pharmaceutically acceptable salt thereof.

2. The compound of claim 1, having the structure: 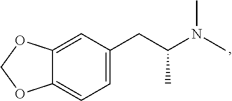
substantially free of a compound of Formula II:

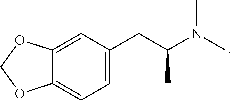

3. The compound of claim 1 or 2, wherein the compound of Formula I comprises hydrochloric acid, hydrobromic acid, hydriodic acid, nitric acid, carbonic acid, monohydrogencarbonic acid, phosphoric acid, monohydrogenphosphoric acid, dihydrogenphosphoric acid, sulfuric acid, monohydrogensulfuric acid, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, glucuronic acid, galactunoric acid, or glutamic acid.

4. The compound of any one of claims 1 to 3, having the structure:

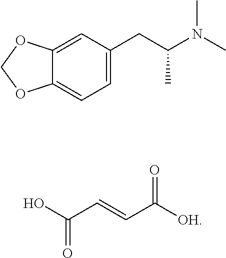

5. An isolated compound of Formula I: 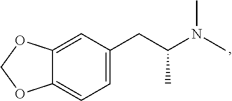
or a pharmaceutically acceptable salt thereof.

6. The isolated compound of claim 5, substantially free of a compound of Formula II:

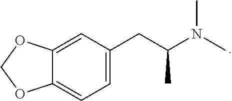

7. A composition comprising
a compound of Formula I: 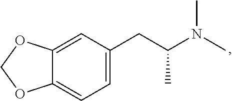
or a pharmaceutically acceptable salt thereof; and
a compound of Formula II:
O N 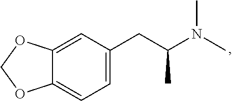
or a pharmaceutically acceptable salt thereof, wherein the compound of Formula II is present in an amount of less than 10% (w/w).

8. The composition of claim 7, wherein the compound of Formula II is present in an amount of less than 1% (w/w).

9. A method for increasing neural plasticity, comprising contacting a neural cell with a compound of any one of claims 1 to 6, or a pharmaceutically acceptable salt thereof, or a composition of claim 7 or 8, in an amount sufficient to increase neural plasticity of the neural cell.

10. The method of claim 9, comprising contacting the neural cell with a compound of Formula I: 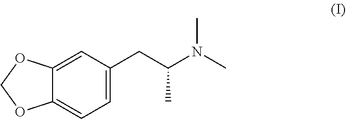
or a pharmaceutically acceptable salt thereof, in an amount sufficient to increase neural plasticity of the neural cell.

11. The method of claim 9 or 10, wherein the compound has the structure:

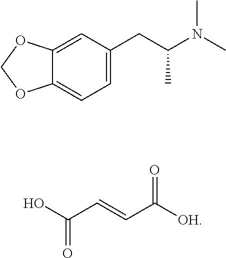

12. A method of treating a brain disorder, comprising administering to a subject in need thereof, a therapeutically effective amount of a compound of any one of claims 1 to 6, or a pharmaceutically acceptable salt thereof, or a composition of claim 7 or 8, thereby treating the brain disorder.

13. The method of claim 12, comprising administering to the subject in need thereof, a therapeutically effective amount of a compound of Formula I: 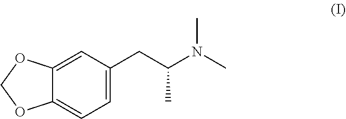
or a pharmaceutically acceptable salt thereof, thereby treating the brain disorder.

14. The method of claim 12 or 13, wherein the compound has the structure:

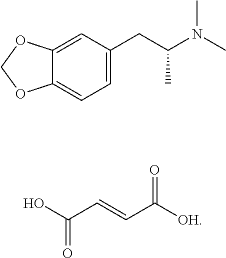

15. The method of any one of claims 12 to 14, wherein the brain disorder is a neurodegenerative disorder, Alzheimer's, or Parkinson's disease.

16. The method of any one of claims 12 to 14, wherein the brain disorder is a psychological disorder, depression, addiction, anxiety, or a post-traumatic stress disorder.

17. The method of claim 16, wherein the brain disorder is one or more of generalized anxiety disorder, phobia, social anxiety disorder, social phobia, panic disorder, panic attack, post-traumatic stress disorders, separation anxiety disorder, selective mutism, agoraphobia, or an anxiety disorder induced by a substance/medication or due to another medical condition.

18. The method of claim 16 or 17, wherein the brain disorder is social anxiety disorder.

19. The method of claim 16 or 17, wherein the brain disorder is post-traumatic stress disorder.

20. The method of claim 16, wherein the brain disorder is depression.

21. The method of claim 16, wherein the brain disorder is addiction.

22. The method of any one of claims 12 to 14, wherein the brain disorder is treatment resistant depression, suicidal ideation, major depressive disorder, bipolar disorder, or substance use disorder.

23. The method of any one of claims 12 to 14, wherein the brain disorder is schizophrenia.

24. The method of any one of claims 12 to 14, wherein the brain disorder is alcohol use disorder.

25. The method of any one of claims 12 to 14, wherein the brain disorder is stroke or traumatic brain injury.

26. The method of any one of claims 12 to 25, comprising administering one or more additional therapeutic agent that is lithium, Olanzapine (Zyprexa), Quetiapine (Seroquel), Risperidone (Risperdal), Ariprazole (Abilify), Ziprasidone (Geodon), Clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

27. A compound of any one of claims 1 to 6, or a pharmaceutically acceptable salt thereof, or a composition of claim 7 or 8, for use in treating a brain disorder.

28. The use of a compound of any one of claims 1 to 6, or a pharmaceutically acceptable salt thereof, or a composition of claim 7 or 8, for the manufacture of a medicament for use in treating a brain disorder.

29. A method for increasing at least one of translation, transcription or secretion of neurotrophic factors, comprising contacting a neural cell with a compound of any one of claims 1 to 6, or a pharmaceutically acceptable salt thereof, or a composition of claim 7 or 8, in an amount sufficient to increase at least one of translation, transcription or secretion of neurotrophic factors.

30. The method of claim 29, comprising contacting the neural cell with a compound of Formula I: 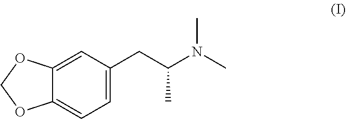
or a pharmaceutically acceptable salt thereof, in an amount sufficient to increase neural plasticity of the neural cell.

31. The method of claim 29 or 30, wherein the compound has the structure:

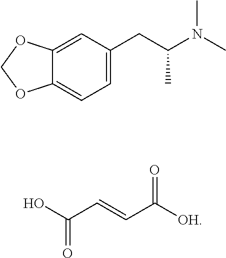

32. A method for modulating a 5-HT2 receptor, comprising contacting a neural cell with a compound of any one of claims 1 to 6, or a pharmaceutically acceptable salt thereof, or a composition of claim 7 or 8, in an amount sufficient to modulate the 5-HT2 receptor.

33. A method for treating a disease or condition responsive to the modulation of a 5-HT2 receptor, comprising contacting a neural cell with a compound of any one of claims 1 to 6, or a pharmaceutically acceptable salt thereof, or a composition of claim 7 or 8, in an amount sufficient to treat the disease or condition responsive to modulation of the 5-HT2 receptor.

34. The method for modulating of claim 32 or 33, wherein the 5-HT2 receptor is at least one of 5-HT2A, 5-HT2B, or 5-HT2C receptor.

35. The method for modulating of claim 32 or 33, wherein the 5-HT2 receptor is the 5-HT2C receptor.
